(19) United States
(12) Patent Application Publication
(10) Pub. No.: US 2021/0161924 A1
(43) Pub. Date: Jun. 3, 2021

(54) NOVEL OLIGOSACCHARIDES FOR USE IN PREBIOTIC APPLICATIONS

(71) Applicant: EVOLVE BIOSYSTEMS, INC., Davis, CA (US)

(72) Inventor: Steven A. FRESE, Oakland, CA (US)

(21) Appl. No.: 17/254,495

(22) PCT Filed: Jun. 19, 2019
(86) PCT No.: PCT/US2019/038062

§ 371 Date: Dec. 21, 2020

Related U.S. Application Data
(60) Provisional application No. 62/687,235, filed on Jun. 19, 2018.

(51) Int. Cl. A61K 31/702; A61K 35/745; A61K 35/747; A61K 35/744; A23L 33/125; A23L 33/135; A23L 33/00; A23K 20/163; A23K 10/18

(52) U.S. Cl. CPC A61K 31/702; A61K 35/745; A61K 35/747; A61K 35/744; A23L 33/125; A23Y 2300/45; A23L 33/40; A23K 20/163; A23K 10/18; A23V 2002/00; A23L 33/135

(57) ABSTRACT

Mare's milk plays a major role in the health of neonatal foals and may be used for human or other mammalian health. Oligosaccharides (OS) possess various bioactivities in many mammalian milks, and Mare's milk may be a source of novel oligosaccharides. The invention relates to the identification of novel structures in mare's milk that may be synthesized or otherwise purified for use in a variety of animal and human applications related to the gut microbiome and mammalian health.

### FIELD OF INVENTION

The invention relates to the identification of novel structures in mare's milk that may be synthesized or otherwise purified for use in a variety of animal and human applications related to the gut microbiome and mammalian health.

### BACKGROUND

Human milk represents the richest known source of free oligosaccharides and are comprised of over 200 different structures (up to date, 247 human milk OS have been separated, 162 of which have been characterized) with a 3-20 g/L concentration [Ward, R.E et al. Molecular nutrition & food research 2007, 51, 1398-1405; Urashima, T.; et al. Trends Glycosci Glyc 2018, 30, 5E51-5E65].

Although oligosaccharides are not digestible by neonates, they exhibit a wide variety of biological roles, with potential prebiotic, antimicrobial, anti-adhesive and immunomodulatory activity. In particular, their ability to promote the growth of beneficial microbes in the gut makes these compounds extremely valuable for health [Bode, L. The Journal of Nutrition 2006, 136, 2127-2130].

### SUMMARY OF INVENTION

This invention describes the structure of seven oligosaccharides found in mare's milk but not in milk from other mammalian species, i.e, these oligosaccharides are uniquely found in mare's milk (see Table 4).

The invention provides methods for preparing novel compositions for horses and/or other mammals. In particular, OS compositions of this invention are tailored for use with mammalian neonates.

The invention provides methods for preparing a nutritive composition for foals and/or other mammalian neonates comprising the steps of obtaining a first composition comprising proteins, lipids, and carbohydrates which are digestible by the neonatal equine gut, and adding one or more oligosaccharides that are uniquely found in equine milk to the first composition. The first composition may comprise or consist of a mammalian milk, which may or not be mare's milk.

The invention also provides a nutritive composition comprising proteins, lipids, and carbohydrates which are digestible by the immature equine gut, plus mammalian milk oligosaccharides that are not digestible by the immature equine gut, and one or more oligosaccharides that are uniquely found in equine milk. In a preferred embodiment, one or more of the mammalian milk oligosaccharides that are not digestible by the immature equine gut are oligosaccharides that are not found in equine milk. This nutritive composition may be used in place of or as a supplement to mare's milk for nutrition of foals.

The invention further provides a nutritive composition for mammalian neonates, especially humans, comprising milk oligosaccharides found in (a) mammalian milk, preferably in human or bovine milk, and/or (b) equine milk. In a preferred embodiment, the milk oligosaccharides are provided by inclusion in, but not limited to human milk, bovine milk, infant formula, follow on formula, weaning foods, baby foods, meal replacers, feeds and feed supplements, beverages, post-surgery recovery drinks, in a powder form, which may optionally be suspended in oil, to be added to conventional foods or beverages. This nutritive composition may provide substantially complete nutrition for the mammalian neonates.

In other embodiments, the composition provides oligosaccharides contemporaneously with a typical diet for the mammal for which the composition is prepared.

The OS identified may be made synthetically to produce structures identical to the composition and mass and/or retention time identified in table 4. In some embodiments, a composition comprising Gal(β1-3)Gal(β1-4)Glc, Gal(β1-6)Gal(β1-4)Glc and/or Gal(β1-3)[Gal(β1-4)GlcNAc(β1-6)]Gal(β1-4)Glc is used in a mammal. In other preferred embodiments, the composition comprising Gal(β1-3)Gal(β1-4)Glc, Gal(β1-6)Gal(β1-4)Glc and/or Gal(β1-3)[Gal(β1-4)GlcNAc(β1-6)]Gal(β1-4)Glc is used in a human or equine application.

In any of the foregoing embodiments, lacto-N-biose (LNB), N-acetyl lactosamine, lacto-N-triose, lacto-N-tetraose (LNT), lacto-N-neotetraose (LNnT), fucosyllactose (FL), lacto-N-fucopentaose (LNFP), lactodifucotetraose, (LDFT) sialyllactose (SL), disialyllacto-N-tetraose (DSLNT), 2′-fucosyllactose (2FL), 3′-sialyllactosamine (3SLN), 3′-fucosyllactose (3FL), 3′-sialyl-3-fucosyllactose(3S3FL), 3′-sialyllactose (3SL), 6′-sialyllactosamine (6SLN), 6′-sialyllactose (6SL), difucosyllactose (DFL), lacto-N-fucopentaose I (LNFPI), lacto-N-fucopentaose II (LNFPII), lacto-N-fucopentaose III (LNFPIII), lacto-N-fucopentaose V (LNFPV), sialyllacto-N-tetraose (SLNT), trifucosyllacto-N-hexaose (TFLNH), LnNH, lacto-N-hexaose (LNH), lacto-N-fucopentaose III (LNFPIII), monofucosylated lacto-N-Hexose III (MFLNHIII), Monofucosylmonosialyllacto-N-hexose (MFMSLNH), galacto-oligosaccharides (GOS, Fructooligosaccharides (FOS), xylooligosaccharide (XOS), their derivatives, or combinations thereof may be included.

In any of the foregoing embodiments, this composition may include a Bifidobacterium and/or Lactobacillus and/or Pediococcus, more preferably B. infantis and L. plantarum or P. acidilactici. Other suitable bacteria are listed below.

The bacteria can be a single bacterial species of Bifidobacterium including but not limited to Bifidobacterium adolescentis, Bifidobacterium animalis, (e.g., Bifidobacterium animalis subsp. animalis, Bifidobacterium animalis subsp. lactis), Bifidobacterium bifidum, Bifidobacterium breve, Bifidobacterium catenulatum, Bifidobacterium longum (e.g., Bifidobacterium longum subsp. suis, Bifidobacterium longum subsp. infantis or Bifidobacterium longum subsp. longum), Bifidobacterium pseudocatanulatum, and Bifidobacterium pseudolongum.

The bacteria can be a single bacterial species of Lactobacillus bacteria, including but not limited to Lactobacillus acidophilus, Lactobacillus antri, Lactobacillus brevis, Lactobacillus casei, Lactobacillus coleohominis, Lactobacillus crispatus, Lactobacillus curvatus, Lactobacillus fermentum, Lactobacillus gasseri, Lactobacillus johnsonii, Lactobacillus mucosae, Lactobacillus pentosus, Lactobacillus plantarum, Lactobacillus reuteri, Lactobacillus rhamnosus, Lactobacillus sakei, Lactobacillus salivarius, Lactobacillus paracasei, Lactobacillus kisonensis., Lactobacillus paralimentarius, Lactobacillus perolens, Lactobacillus apis, Lactobacillus ghanensis, Lactobacillus dextrinicus, Lactobacillus shenzenensis, Lactobacillus harbinensis, and Lactobacillus equi.

The bacteria can be a single bacterial species of Pediococcus bacteria, including but not limited to Pediococcus parvulus, Pediococcus lolii, Pediococcus acidilactici, Pediococcus argentinicus, Pediococcus claussenii, Pediococcus pentosaceus, or Pediococcus stilesii.

The bacteria can include, but are not limited to, one or more of B. adolescentis, B. animalis, (e.g., B. animalis subsp. animalis or B. animalis subsp. lactis), B. bifidum, B. breve, B. catenulatum, B. longum (e.g., B. longum subsp. infantis or B. longum subsp. longum or B. longum subsp. suis), B. pseudocatanulatum, and B. pseudolongum that demonstrate the capability of consuming MMO.

In yet other embodiments, the composition comprises at least 2 different species, or at least 3 different species selected from any of the aforegoing species.

In any of the foregoing embodiments, the composition may comprise bacteria in an amount of 0.1 million-500 billion Colony Forming Units (CFU) per gram of composition. The composition may comprise bacteria may be in an amount of 0.001-100 billion Colony Forming Units (CFU) 0.1 million to 100 million, 1million to 5 billion, or 5-20 billion Colony Forming Units (CFU) per gram of composition. The bacteria may be in an amount of 0.001, 0.01, 0.1, 1, 5, 15, 20, 25, 30, 35, 40, 45, or 50 billion Colony Forming Units (CFU) per gram of composition. The bacteria may be in an amount of 5-20 billion Colony Forming Units (CFU) per gram of composition or 5-20 billion Colony Forming Units per gram of composition or 0.1 million to 100 million Colony Forming Units per gram of composition. The bacteria may be given once daily either together or separate from the OS composition.

While the preceding has largely focused on human and equine neonates it should be understood to apply to any mammal, such as but not limited to cow, goat, sheep, camel, elephant, dog, cat of any age including a mammalian neonate. The mammal may have a disease or condition requiring treatment or the compositions may be used as part of a mammalian diet to prevent or otherwise keep the mammal healthy.

### BRIEF DESCRIPTION OF FIGURES

FIG. 1. Variation of mare's milk OS type (neutral, fucosylated, acidic) during the first week of lactation by Nano LC Chip QTOF MS. Results are expressed as the average±standard deviation (n=4) of the relative abundance of each class of OS in milk from four animals.

FIG. 2. Variation of key OS in mare's milk during the first week of lactation measured by Nano LC Chip QTOF MS. Results are expressed as the average±standard deviation (n=4) of the relative abundance of each class of OS.

FIG. 3. Evolution of LNT/LNnT and LNH/LNnH during lactation analyzed by nano LC-Chip QToF-MS.

FIG. 4. HPAEC-PAD chromatograms of OS in four mare milks at different lactation stages. (a) Day 0, (b) Day 7. Peak 1, 2Hex-1HexNAc; Peak 2, LNnT; Peak 3, 3 hexose; Peak 4, LNT; Peak 5, 3′SLN; Peak 6, 6′SL; and Peak 7, 3′SL.

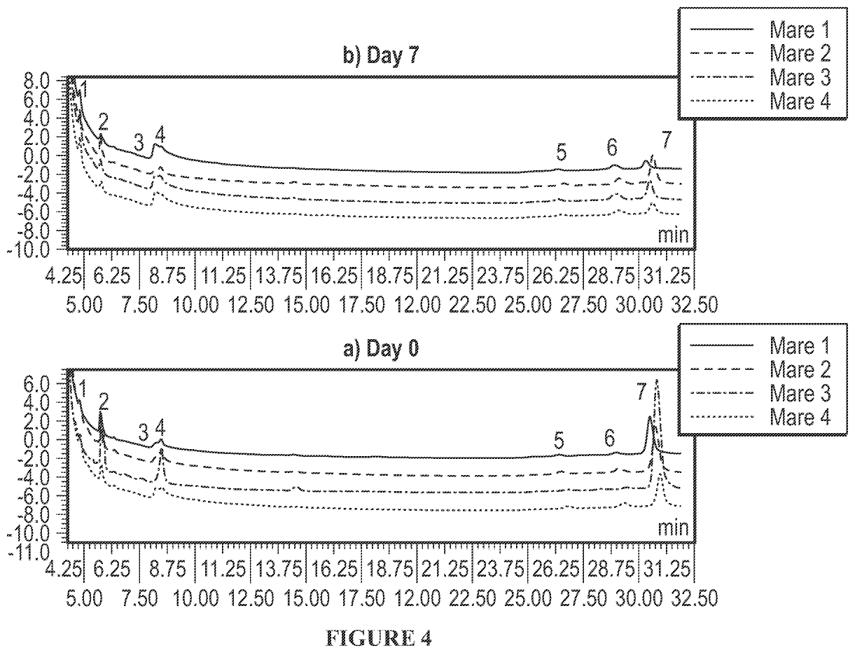

FIG. 5. OS standard (0.001 g/L) chromatogram obtained by HPAEC-PAD. Peak 1, 2Hex-1HexNAc; Peak 2, LNnT; Peak 3, 3 Hexose; Peak 4, LNT; Peak 5, LNnH; Peak 6, LNH; Peak 7, 6′SLN; Peak 8, 3′SLN; Peak 9, 6′SL and Peak 10, 3′ SL.

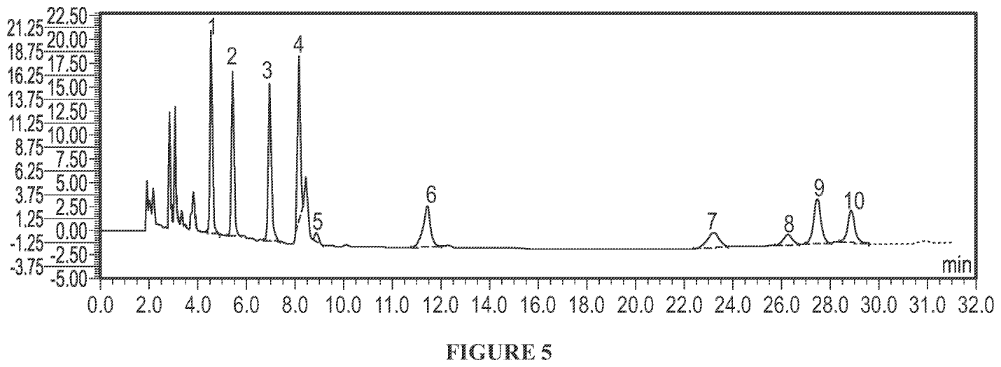

### DETAILED DESCRIPTION OF THE INVENTION

Depending on the species, milk oligosaccharides (OS) are typically composed of 3-20 monosaccharide units, including glucose (Glc), galactose (Gal), N-acetyl-glucosamine (GlcNAc), N-acetyl-galactosamine (GalNAc), fucose (Fuc) and sialic acids (NeuAc/ NeuGc). Their core units can be either lactose [Gal(β1-4)Glc] or lactosamine [Gal(β1-4)GlcNAc]. Based on chemical composition, OS are classified as neutral (containing glucose/galactose/GlcNAc/GalNAc/fucose) or acidic (which include the previously mentioned monosaccharides and are further decorated by the sialic acids NeuAc/NeuGc). [Aldredge, et al. Glycobiology 2013, 23, 664-676]. In more preferred embodiments, the OS are 3-10 monosaccharide units. Oligosaccharides called glycans may be bound to proteins or lipids. When released from the protein or lipid they may contribute to the pool of oligosaccharides.

In some embodiments, the structural composition determines the accessibility of carbohydrates for bacteria in the large intestine and selects which taxa dominate the distal gut of neonates. In yet other embodiments, the oligosaccharides limit the growth of pathogens in the gut and may also reduce stool pH. In even further embodiments, addition of the oligosaccharide structures can alter the growth of the host animal including but not limited to weight gain, distribution of weight gain including lean to fat mass, and vitamin status, etc. Oligosaccharides having the chemical structure of the indigestible oligosaccharides found in any mammalian milk are called OS herein, whether or not they are actually sourced from mammalian milk. They may be sources from bacterial, fungal, insect, algae or other enzymatic or chemical production systems known in the art for production of compounds.

The term “synthetic” composition refers to a composition produced by a chemi-synthetic process and can be nature-identical. For example, the composition can include ingredients that are chemically synthesized and purified for use in the composition. The OS may be produced in a production system such as yeast or E. coli. The OS composition may be purified, enriched or isolated from natural equine sources or it may be a combination of both to make a composition that is synthetic to be used in this invention.

Purification of the oligosaccharide can mean separating a component of milk from any other components in milk or otherwise processing mammalian milk including expressing human milk to provide for example the foremilk which is partially skimmed, human donor milk, or other human milk products such as fortifiers.

In some embodiments, a total OS fraction is purified from mare's milk wherein an expected total of 48 OS structures (including isomers and anomers), may be identified by nano-LC-Chip QTOF-MS and confirmed by tandem mass spectrometry. These structures may correspond to 20 unique compositions. In preferred embodiments, the Neutral OS are 40-60% of the total oligosaccharides (e.g., 58.3%), acidic OS containing Neu5Ac 20-40% (e.g., 33.3%), fucosylated OS structures 2-10% (e.g., 6.25%) and one structure containing NeuGc 1-5% (e.g., 2.1%). In some embodiments, the the percentage of neutral and fucosylated OS increased to greater than 60% and greater than 10% respectively and acidic OS decreased below 20%.

In some embodiments, the OS is selected from the group listed here and also listed in Table 4: 2Hex, 2HexNAc (mass 750.2907, RT 24 min); 2Hex, 1HexNAc 1Neu5Ac (mass 838.3062 , RT 21.5 min), 2Hex, 1HexNAc 1Neu5Ac (mass 838.3062, RT 19.67 min); 5Hex 1Neu5Ac (mass 976.3479, RT; 2Hex 2HexNAc 1 Neu5Ac (mass 1041.386, RT 23.25 min); 2Hex 2HexNAc 1 Neu5Ac (mass 1041.386, RT 24.36 min); 1Hex 1HexNAc 1 Fuc (mass 531.2159, RT 26.05 min). These structures may be measured by Nano LC-Chip QTOF MS.

Any of the foregoing compositions may additionally comprise 2 sialyllactose isomers, 3 Hexose, Lacto-N-Hexaose, Lacto-N-tetraose (LNT), Lacto-N-neohexaose(LNnH), Lacto-N-neotetraose (LNnT), and OS with the composition 3 Hex-1 Neu5Ac).

In any of the foregoing embodiments, lacto-N-biose (LNB), N-acetyl lactosamine, lacto-N-triose, lacto-N-tetraose (LNT), lacto-N-neotetraose (LNnT), fucosyllactose (FL), lacto-N-fucopentaose (LNFP), lactodifucotetraose, (LDFT) sialyllactose (SL), disialyllacto-N-tetraose (DSLNT), 2′-fucosyllactose (2FL), 3′-sialyllactosamine (3SLN), 3′-fucosyllactose (3FL), 3′-sialyl-3-fucosyllactose(3S3FL), 3′-sialyllactose (3SL), 6′-sialyllactosamine (6SLN), 6′-sialyllactose (6SL), difucosyllactose (DFL), lacto-N-fucopentaose I (LNFPI), lacto-N-fucopentaose II (LNFPII), lacto-N-fucopentaose III (LNFPIII), lacto-N-fucopentaose V (LNFPV), sialyllacto-N-tetraose (SLNT), trifucosyllacto-N-hexaose (TFLNH), LnNH, lacto-N-hexaose (LNH), lacto-N-fucopentaose III (LNFPIII), monofucosylated lacto-N-Hexose III (MFLNHIII), Monofucosylmonosialyllacto-N-hexose (MFMSLNH), galacto-oligosaccharides (GOS), Fructooligosaccharides (FOS), xylooligosaccharide (XOS), their derivatives, or combinations thereof may be included.

The oligosaccharides may include: (a) one or more Type II oligosaccharide core where representative species include LnNT; (b) one or more oligosaccharides containing the Type II core and GOS in 1:5 to 5:1 ratio; (c) one or more oligosaccharides containing the Type II core and 2FL in 1:5 to 5:1 ratio; (d) a combination of (a), (b), and/or (c); (e) one or more Type I oligosaccharide core where representative species include LNT (f) one or more Type I core and GOS in 1:5 to 5:1 ratio; (g) one or more Type I core and 2FL in 1:5 to 5:1 ratio; and/or (h) a combination of any of (a) to (g) that includes both a type I and type II core. Type I or type II may be isomers of each other. Other type II cores include but are not limited to trifucosyllacto-N-hexaose (TFLNH), LnNH, lacto-N-hexaose (LNH), lacto-N-fucopentaose III (LNFPIII), monofucosylated lacto-N-Hexose III (MFLNHIII), Monofucosylmonosialyllacto-N-hexose (MFMSLNH). In some embodiments, glycans released from 0-linked or N-linked glycoproteins or glycolipids may be used as part of the total OS concentration.

In some embodiments, the total OS concentration may range from 20 milligrams/Liter to 40 grams/Liter. In some embodiments, the OS may be in the range 20-80 mg/L, 60-200, mg/L, 80-220 mg/L, 220-500 mg/L, 500 mg/L- 1000mg/L, 1-4 gram/L., 4 -12 g/L, 12-25g/L and 25-40 g/L. In some embodiments, the OS is represented by a single OS structure, including but not limited to those in table 4. In other embodiments, the OS concentration is made up of more than a single structure.

In some embodiments, the OS profile of different mammalian milks may be used for targeted product development that is species-specific, or specific to certain diseases or conditions.

Compositions of the invention may include one or more species of bacteria. Suitable bacteria are listed below.

The bacteria can be a single bacterial species of Bifidobacterium including but not limited to Bifidobacterium adolescentis, Bifidobacterium animalis, (e.g., Bifidobacterium animalis subsp. animalis, Bifidobacterium animalis subsp. lactis), Bifidobacterium bifidum, Bifidobacterium breve, Bifidobacterium catenulatum, Bifidobacterium longum (e.g., Bifidobacterium longum subsp. suis, Bifidobacterium longum subsp. infantis or Bifidobacterium longum subsp. longum), Bifidobacterium pseudocatanulatum, Bifidobacterium pseudolongum.

The bacteria can be a single bacterial species of Lactobacillus bacteria, including but not limited to Lactobacillus acidophilus, Lactobacillus antri, Lactobacillus brevis, Lactobacillus casei, Lactobacillus coleohominis, Lactobacillus crispatus, Lactobacillus curvatus, Lactobacillus fermentum, Lactobacillus gasseri, Lactobacillus johnsonii, Lactobacillus mucosae, Lactobacillus pentosus, Lactobacillus plantarum, Lactobacillus reuteri, Lactobacillus rhamnosus, Lactobacillus sakei, Lactobacillus salivarius, Lactobacillus paracasei, Lactobacillus kisonensis., Lactobacillus paralimentarius, Lactobacillus perolens, Lactobacillus apis, Lactobacillus ghanensis, Lactobacillus dextrinicus, Lactobacillus shenzenensis, Lactobacillus harbinensis, Lactobacillus equi.

The bacteria can be a single bacterial species of Pediococcus bacteria, including but not limited to Pediococcus parvulus, Pediococcus lolii, Pediococcus acidilactici, Pediococcus argentinicus, Pediococcus claussenii, Pediococcus pentosaceus, or Pediococcus stilesii.

In any of the above embodiments, the bacteria can be Bifidobacterium longum subsp. infantis EVC001 as deposited under ATCC Accession No. PTA-125180; cells were deposited with the American Type Culture Collection at 10801 University Blvd, Manassas, Va. 20110 under the Budapest Treaty on the International Recognition of the Deposit of Microorganisms for the Purposes of Patent Procedure, the “Deposited Bacteria.”

Additionally, “Deposited Bacteria,” as used herein, refers to the isolated Bifidobacterium longum subsp. infantis EVC001, deposited with the ATCC and assigned Accession Number, and variants thereof, wherein said variants retain the phenotypic and genotypic characteristics of said bacteria and wherein said bacteria and variants thereof have LNT transport capability and comprise a functional H5 gene cluster comprising at least BLON2175, BLON2176, and BLON2177, described in International Application PCT/US2019/034765, filed May 30, 2019.

A “functional H5 cluster,” refers to a cluster of genes in Bifidobacterium responsible for the uptake and metabolism of human milk oligosaccharides. A functional H5 cluster comprises Blon_2175, Blon_2176, and Blon_2177. The H5 cluster comprises the following genes: Blon_2171, Blon_2173, Blon_2174, Blon_2175, Blon_2176, Blon_2177, and galT.

Activation is defined as a means of turning on a specific nutrient consumption phenotype (like the HMO phenotype in B. infantis) in bacteria during production of the bacteria, which are dried in that state, examples of which are included in International Patent Application Nos. PCT/US2015/057226, filed Oct. 23, 2015, and PCT/US2019/014097, filed Jan. 18, 2019.

In any of the foregoing embodiments, the composition may be formulated as a capsule, packet, sachet, foodstuff, lozenge, tablet, optionally an effervescent tablet, enema, suppository, dry powder, dry powder suspended in an oil, chewable composition, syrup, liquid or gel in a shelf stable format. That may mean frozen, refrigerated or room temperature.

The nutritional product may be a food product, dietary supplement, infant formula, or pharmaceutical product.

In a preferred embodiment, the milk oligosaccharides are provided by inclusion in, but not limited to human milk, bovine milk, infant formula, follow on formula, weaning foods, baby foods, meal replacers, feeds and feed supplements, beverages, post-surgery recovery drinks. These compositions may be added to conventional foods or beverages at the time of ingestion or incorporated during production or packaging of the conventional food or beverage. In particular, compositions comprising bacteria may be in a powder form, which may optionally be suspended in oil or other anhydrous environments in a shelf stable format. Any of the compositions can be made as a powder. This nutritive composition may provide substantially complete nutrition for the mammal.

The mammal may be in need of increased abundance of beneficial commensal organisms such as Bifidobacterium; Lactobacillus or Pediococcus in their intestines. The mammal may require any of the compositions to reduce the abundance of pathogens or potential pathogens. The mammal may use any of the compositions to reduce stool pH or reduce diarrhea associated with an infection or disease condition. The compositions may be used to reduce diaper rash, colic or improve sleep.

The compositions of this invention may be administered for at least 24 hours, at least 72 hours, at least 21 days, at least 28 days, at least 12 weeks, 16 weeks, 6 months, or at least 1 year to develop a robust and appropriate immune modification.

The treatment may be designed to stimulate the immune system for the purpose of improving host defense, including but not limited to improving mucus production and/or reducing mucus degradation, B cell responsiveness and/or expanding or altering the T Regulatory and Helper T cell profile. The composition may result in induction of oral tolerance and improved vaccine efficacy.

The mammal may use any of the composition to prevent or treat a number of different autoimmune conditions, inflammatory conditions, and therapies requiring a functioning immune system may be improved by use of the compositions described herein including, but not limited to, inflammatory bowel disease (IBD) including Crohn's disease and ulcerative colitis and inflammatory bowel syndrome (IBS), necrotizing enterocolitis colitis (NEC), allergy, atopy, celiac, obesity, Type 1 Diabetes, Type II diabetes, vaccine responsiveness, autism, organ transplant, immunotherapy, and gene therapy.

Treatment Protocol

This invention provides methods for treating a dysbiotic mammal by: (i) administering a bacterial composition comprising bacteria capable of and/or activated for colonization of the colon; (ii) administering a composition comprising OS; or (iii) both (i) and (ii) added contemporaneously. This embodiment can alternatively provide a method of enhancing the health of a mammal. The bacteria and/or the OS composition can be administered in respective amounts sufficient to achieve the health enhancement or therapeutic effect desired.

Generally, the phrase “dysbiosis” describes a non-ideal state of the microbiome inside the body, typified as an insufficient level of keystone bacteria (e.g., bifidobacteria, such as B. longum subsp. infantis) or an overabundance of harmful bacteria in the gut. Dysbiosis can be further defined as inappropriate diversity or distribution of species abundance for the age of the human or animal. Dysbiosis may also refer to the abundance of specific gene functions, such as, but not limited to abundance of antibiotic resistance genes in the microbiome.

Bacteria that selectively grows on the OS can be provided contemporaneously with the MMO, or the bacteria can be provided separately to a nursing infant whose OS are in the form of whole milk provided by nursing or otherwise, or the bacteria and the OS may be provided in the same composition. A composition comprising from 0.1 million to 500 billion cfu of bacteria can be provided on a daily basis, or the composition may comprise from 1 billion to 100 billion cfu, or from 5 billion to 20 billion cfu provided on a daily basis. The OS can be provided in a solid or liquid form at a dose from about 0.1-50 g/day, for example, 2-30 g/day or 3-10 g/d.

The bacteria and the OS can be provided contemporaneously or separately at any time during 24 hours. The OS could for example be provided along with an infant formula and the bacteria provided separately within 24 hr, 12 hr, 8 hr, 6 hr, 4hr or 2hr of consumption of the OS. In certain embodiments of the instant invention, a “daily ration” of the bacteria and OS is provided to the patient. A “daily ration” is an amount provided to the patient within the same 24-hour period. A patient can be given a dose of the bacteria and a dose of the OS substantially contemporaneously (e.g., within six hours, within four hours, within two hours, within one hour, within forty-five minutes, within thirty minutes, within twenty minutes, within fifteen minutes, within ten minutes, within five minutes, within three minutes, or within one minute). The dose of OS in the daily ration may be provided in a single administration or in a plurality of administrations (twice per day, three times per day, etc.), and the dose of bacteria may be provided separately or at the same time as one of the OS administrations.

In another embodiment, the bacteria is administered during an initial treatment period, and then discontinued after 2 days, one week, 2 weeks, etc., while the OS is administered continuously from the initiation of treatment for multiple months up to four months, six months or more. The OS can be provided in its own composition or as part of a food composition. The food composition can include mammalian milk, mammalian milk derived product, mammalian donor milk, an infant formula, milk replacer, or enteral nutrition product, or meal replacer for a mammal including a human, and the food composition can include the complete nutritional requirements to support life of a healthy mammal wherein that mammal may be, but is not limited to, an infant, an adolescent, an adult, or a geriatric adult. The mammal may be, but not limited to, a human, horse, cow, sheep, goat, camel, dog, cat, llama, or elephant. It may be a neonate of any of the above mammals. It may also be a mammal of any age who needs any of the compositions disclosed herein.

### EXAMPLE 1

### Characterization of Thoroughbred mare's Milk Oligosaccharides

In this example the oligosaccharide content of milk collected from four Thoroughbred mares was analyzed and described over the first week of lactation.

### Materials and Methods

All solvents used for sample preparation were HPLC-MS grade (Fisher Scientific, Fair Lawn, N.J.). Non-porous graphitized carbon solid-phase extraction (GCC-SPE) (2000 μg binding capacity) was purchased from Glygen Corp. (Columbia, Md., USA). Oligosaccharide standards with a minimum purity of 95% (lacto-N-tetraose, LNT; lacto-N-neotetraose, LNnT; lacto-N-hexaose, LNH; lacto-N-neohexaose, LNnH; acetylgalactosaminyl-α1,3-galactose-β-1,4-glucose, 2 Hex—1 HexNAc; galactose-α-1,3-galactose-β-1,4-glucose, 3 Hex; 6′-sialyllactosamine, 6′-SLN; 3′-sialyllactosamine, 3′- SLN; 6′-sialyllactose, 6′- SL; 3′-sialyllactose, 3′-SL) were purchased from V-Labs (Covington, La., USA). Nanopure water (18.2M Ω.cm, 25° C.) was used for the analytical work.

### Sample Collection and Oligosaccharide Isolation and Purification

Thoroughbred mare's milk samples were obtained from a commercial Thoroughbred breeding facility (Vacaville, Calif., USA). Samples were collected daily from four mares over the first week of lactation and stored at −20° C. until analysis. Milk OS were isolated and purified. Briefly, frozen milk samples were completely thawed, and a 0.5-mL aliquot of each sample was mixed with an equal volume of nanopure water and centrifuged at 14,000×g in a microfuge for 30 min at 4° C. to remove lipids. The top fat layer was removed, and 4 volumes of chloroform/methanol (2:1, vol/vol) were added, vigorously mixed and the resulting emulsion was centrifuged at 4,000×g for 30 min at 4° C. The upper methanol layer containing OS was transferred to new tubes, and two volumes of cold ethanol were added. The water-ethanol solution was frozen for 1 h at −30° C., followed by centrifugation for 30 min at 4,000×g and 4° C. to precipitate the denatured protein. The supernatant (OS-rich fraction) was collected and freeze-dried using a speed vacuum centrifuge.

For nano-LC-Chip QTOF-MS analysis, OS were reduced with NaBH4 1M for 1h at 60° C. Once reduced, they were purified from the mixture by solid-phase extraction using nonporous graphitized carbon cartridges (GCC-SPE). Prior to use, each GCC-SPE cartridge was activated with 3 column volumes of 80% acetonitrile (ACN), 0.1% trifluoroacetic acid (TFA, v/v) and equilibrated with 3 column volumes of nanopure water. The carbohydrate-rich solution was loaded onto the cartridge, and salts and mono/disaccharides were removed by washing with 10 column volumes cv of nanopure water. The OS were eluted with a solution of 40% ACN with 0.1% TFA (v/v) in water and dried in a speed vacuum centrifuge at 35° C. overnight.

Oligosaccharides Characterization by Nano LC Chip QTOF MS

Prior to analysis by Nano LC Chip QTOF MS, dried OS samples were reconstituted in 100 μL of nanopure water. MS analysis was performed with an Agilent 6520 accurate-mass Quadrupole-Time-of-Flight (Q-TOF) liquid chromatography/mass spectroscopy (LC/MS) equipped with a micro-fluidic nano-electrospray chip (Agilent Technologies, Santa Clara, Calif., USA) as previously described [23]. The micro-fluidic chip contained one enrichment and one analytical column, both packed with graphitized carbon. Chromatographic elution was performed with a binary gradient of 3% ACN/0.1% formic acid in water (solvent A) and 90% ACN/0.1% formic acid in water (solvent B). The column was initially equilibrated with a flow rate of 0.3 μL/min for the nanopump and 4 μL/min for the capillary pump. The 65-min gradient was programmed as follows: 0-2.5 min, 0% B; 2.5-20 min, 0-16% B; 20-30 min, 16-44% B; 30-35 min, 44-100% B; 35-45 min, 100% B; and 45-65 min, 0% B. Data were acquired in the positive ionization mode with a 450-2500 mass/charge (m/z) range. The electrospray capillary voltage was 1700-1900 V. The acquisition rate was 0.63 spectra/s for both MS and MS/MS modes. Automated precursor selection was employed based on ion abundance, performing up to 6 MS/MS spectra per individual MS when precursor was above ion abundance threshold. The precursor isolation window was selected to be narrow (1.3 m/z) to improve accuracy. Fragmentation energy was set at 1.8 V/100 Da with an offset of −2.4 V. Internal calibration was continuously performed by infusing two reference masses: 922.009 and 1221.991 m/z (ESI-TOF Tuning Mix G1969-85000, Agilent Technologies). To minimize instrumental variation, diluted samples were spiked with 5 μL of 2-fucosyllactose 0.02 g/L, and the results for each OS were normalized against this internal standard.

### Nano LC Chip QTOF Data Analysis

A list of deconvoluted masses in a range of 450-1500 m/z and corresponding to OS was obtained, with all OS compositions confirmed by tandem MS (MS/MS) analysis. The allowed charge states were restricted to single and double species. Following MS/MS identity validation and assessment of reproducible retention times (RT), individual peaks for each OS were automatically integrated using the Targeted Feature Extractor from MassHunter Profinder Version B.06.00 (Agilent Technologies). The RT window allowed for compound matching was restricted to ±0.5 min and ±0.25% of the RT at each time point.

Lactose and Oligosaccharides Quantification by High-Performance Anion-Exchange Chromatography Coupled with Pulsed Electrochemical Detection (HPAEC-PAD)

Extracted and purified OS were re-dissolved in 1 mL of distilled water and diluted 10- to 100-fold in distilled water, filtered through a 0.22 μm membrane. Aliquots of 25 μL at were injected for each analysis. The instrument was equipped with two chromatographic systems allowing simultaneous quantification of lactose and OS quantification; the sample diverted to the correspondent system by a valve installed in the injector system. The chromatographic separation for OS was carried out with a CarboPacPA200 analytical column (3×250 mm, Dionex) and a CarboPacPA200 Guard Column (3×50 mm, Dionex), eluting at 0.5 mL/min with a non-isocratic gradient: 0-10 min 50% B, 10-50 min 45% B-10% C. When lactose was quantified, a CarboPacPA10 analytical column (4×250 mm, Dionex) and a CarboPacPA10 Guard Column (2×50 mm, Dionex) were used, eluting at 1.2 mL/min with a non-isocratic gradient: 0-12 min 5% B, 10-25 min 50% B. For both determinations, the columns were equilibrated 5 min with 10% B followed by 10 min with 50% B. Solvent A was deionized water, solvent B 200 mM NaOH and solvent C was 100 mM NaAc in 100 mM NaOH.

Simultaneous separation and quantification of 10 different OS were carried out by external calibration ranging from 0.0001 to 0.03 g/L. From the total of OS quantified, 6 were neutral (lacto-N-tetraose (LNT), lacto-N-neotetraose (LNnT), lacto-N-hexaose (LNH), lacto-N-neohexaose (LNnH), acetylgalactosaminyl-α-1,3-galactose-β-1,4-glucose (2 Hex-1 HexNAc), galactose-α-1,3-galactose-β-1,4-glucose (3 Hex)) and four were acidic (6′-sialyllactosamine (6′- SLN), 3′-sialyllactosamine (3′-SLN), 6′-sialyllactose (6′- SL) and 3′-sialyllactose (3′-SL)). All samples were analyzed in triplicate.

### Statistical Analysis

A one-way ANOVA was performed using SPSS software (SPSS v23.0.0) to evaluate differences in the relative proportion of OS and individual concentrations of OS in mare's milk throughout the first week of lactation. Prior to statistical analysis, normality and homoscedasticity of the data were checked using the Kolmogorov-Smirnov and Levene tests, respectively; all data were normally distributed and no outliers were identified. Data are presented as least squares means for each lactation time-point. Statistical significance was considered when P<0.05.

### Results

### Characterization of Mare's Milk Oligosaccharides

Although different techniques have been used for identifying OS in milk and other biological samples, Nano LC-Chip QTOF MS/MS is one of the most widely adopted technique because of its inherent accuracy and sensitivity and the ability to resolve multiple isomers for each OS without the need for chemical derivatization. The present study identified and quantified OS in mare's milk during the first week of lactation. A total of 48 structures, including isomers and anomers, of OS corresponding to 20 compositions were detected and confirmed by MS/MS in Thoroughbred mare's milk over the first 7 days of lactation (Table 1).

TABLE 1
Composition and relative abundance of OS in the mare's
milk samples analyzed. Relative abundance (%) is expressed as the average of values
for milk from four mares, each analyzed in triplicate. Composition is reported as
Hex, hexose; HexNAc, N-acetylhexosamine; Fuc: fucose; Neu5Ac: N-
acetylneuraminic acid; and Neu5Gc, N-acetylglycolylneuraminic acid. RT is
retention time in liquid chromatography. Note: OS m/z 531.2159 is double charged.

| | | | | | Exact | | | | | | | | | |
|---|---|---|---|---|---|---|---|---|---|---|---|---|---|---|
| Oligosaccharide composition | | | | | mass | Exact | | OS relative abundance (%) | | | | | | |
| Hex | HexNac | Fuc | Neu5Ac | Neu5Gc | (calc) | mass | RT | Day 1 | Day 2 | Day 3 | Day 4 | Day 5 | Day 6 | Day 7 |
| 3 | 0 | 0 | 0 | 0 | 506.1848 | 506.1854 | 10.49 | 1.0215 | 1.4308 | 1.2059 | 0.9547 | 0.9729 | 0.8388 | 0.7089 |
| 3 | 0 | 0 | 0 | 0 | 506.1848 | 506.1856 | 13.94 | 4.4181 | 5.4741 | 5.3407 | 5.6773 | 5.1573 | 5.2809 | 5.5818 |
| 3 | 0 | 0 | 0 | 0 | 506.1848 | 506.1867 | 25.48 | 0.1253 | 0.2190 | 0.1604 | 0.1410 | 0.1410 | 0.1620 | 0.1980 |
| 3 | 0 | 0 | 0 | 0 | 506.1848 | 506.1873 | 15.89 | 0.1483 | 0.0595 | 0.0748 | 0.0394 | 0.0752 | 0.0554 | 0.0471 |
| 2 | 1 | 0 | 0 | 0 | 547.2113 | 547.2099 | 13.69 | 0.1816 | 0.1298 | 0.1058 | 0.1055 | 0.1192 | 0.0908 | 0.0820 |
| 2 | 1 | 0 | 0 | 0 | 547.2113 | 547.2118 | 14.96 | 0.3442 | 0.3945 | 0.3878 | 0.3928 | 0.3572 | 0.3581 | 0.3234 |
| 2 | 1 | 0 | 0 | 0 | 547.2113 | 547.2122 | 14.37 | 0.5536 | 0.1272 | 0.1126 | 0.1159 | 0.0995 | 0.0715 | 0.0659 |
| 2 | 1 | 0 | 0 | 0 | 547.2113 | 547.2145 | 11.14 | 0.1221 | 0.0312 | 0.0278 | 0.0184 | 0.0278 | 0.0173 | 0.0191 |
| 2 | 0 | 0 | 1 | 0 | 635.2274 | 635.2281 | 18.94 | 21.5480 | 17.7825 | 14.6935 | 14.6102 | 15.8106 | 15.3802 | 15.3318 |
| 2 | 0 | 0 | 1 | 0 | 635.2274 | 635.2328 | 12.42 | 0.0474 | 0.1397 | 0.1288 | 0.1725 | 0.1976 | 0.3237 | 0.3078 |
| 2 | 0 | 0 | 0 | [symbol] | 651.2218 | 651.228 | 18.3 | 0.0734 | 0.0483 | 0.0231 | 0.0273 | 0.0265 | 0.0157 | 0.0143 |
| 4 | 0 | 0 | 0 | 0 | 668.2376 | 668.2384 | 12.77 | 0.6473 | 0.3418 | 0.3173 | 0.2528 | 0.0156 | 0.1814 | 0.1424 |
| 4 | 0 | 0 | 0 | 0 | 668.2376 | 668.2388 | 14.42 | 0.8927 | 0.8751 | 0.7190 | 0.8527 | 0.7044 | 0.5164 | 0.4751 |
| 4 | 0 | 0 | 0 | 0 | 668.2376 | 668.241 | 16.07 | 0.2425 | 0.0610 | 0.0989 | 0.0563 | 0.1094 | 0.0459 | 0.0387 |
| 1 | 1 | 0 | 1 | 0 | 676.2534 | 676.254 | 19.49 | 2.3584 | 0.0951 | 0.0907 | 0.0827 | 0.1292 | 0.0697 | 0.0504 |
| 3 | 1 | 0 | 0 | 0 | 709.2642 | 709.2643 | 17.98 | 0.9538 | 0.8397 | 0.9565 | 0.9781 | 0.9082 | 0.8503 | 0.8014 |
| 3 | 1 | 0 | 0 | 0 | 709.2642 | 709.2645 | 24.87 | 1.5970 | 0.8472 | 0.6428 | 0.5422 | 0.5786 | 0.4353 | 0.3735 |
| 3 | 1 | 0 | 0 | 0 | 709.2642 | 709.2645 | 14.96 | 1.0818 | 1.3143 | 1.3313 | 1.3031 | 1.2064 | 1.1786 | 1.0591 |
| 3 | 1 | 0 | 0 | 0 | 709.2642 | 709.2652 | 11.84 | 12.9499 | 12.9067 | 10.8475 | 9.9517 | 9.9498 | 8.3908 | 6.9171 |
| 3 | 1 | 0 | 0 | 0 | 709.2642 | 709.2657 | 14.2 | 11.3807 | 19.6812 | 25.4998 | 27.0419 | 27.3783 | 31.0953 | 33.0486 |
| 2 | 2 | 0 | 0 | 0 | 750.2907 | 750.2853 | 16.16 | 0.1671 | 0.0736 | 0.0760 | 0.0812 | 0.0733 | 0.0743 | 0.0745 |
| 2 | 2 | 0 | 0 | 0 | 750.2907 | 750.2885 | 13.53 | 0.5731 | 0.1958 | 0.1519 | 0.1392 | 0.1656 | 0.1254 | 0.1452 |
| 2 | 2 | 0 | 0 | 0 | 750.2907 | 750.2907 | 24 | 0.2206 | 0.0236 | 0.0070 | 0.0063 | 0.0110 | 0.0025 | 0.0000 |
| 2 | 2 | 0 | 0 | 0 | 750.2907 | 750.2911 | 19.55 | 0.0580 | 0.0866 | 0.0757 | 0.0774 | 0.0597 | 0.0729 | 0.0694 |
| 2 | 2 | 0 | 0 | 0 | 750.2907 | 750.2911 | 12.6 | 0.6886 | 0.2472 | 0.4056 | 0.4498 | 0.5382 | 0.6418 | 0.6943 |
| 3 | 0 | 0 | 1 | 0 | 797.2802 | 797.2804 | 21.51 | 1.3695 | 0.8747 | 0.6561 | 0.6153 | 0.6314 | 0.4256 | 0.4442 |
| 3 | 0 | 0 | 1 | 0 | 797.2802 | 797.2805 | 19.44 | 0.4063 | 0.1417 | 0.1680 | 0.1757 | 0.1207 | 0.1403 | 0.1271 |
| 5 | 0 | 0 | 0 | 0 | 830.2905 | 830.2912 | 13.42 | 3.1050 | 0.3951 | 1.8426 | 0.1885 | 1.8018 | 0.2039 | 0.1326 |
| 2 | 1 | 0 | 1 | 0 | 838.3062 | 838.3057 | 21.98 | 1.5229 | 0.0042 | 0.0000 | 0.0016 | 0.0153 | 0.0000 | 0.0000 |
| 2 | 1 | 0 | 1 | 0 | 838.3062 | 838.3061 | 21.5 | 1.6260 | 0.0037 | 0.0000 | 0.0007 | 0.0153 | 0.0000 | 0.0000 |
| 2 | 1 | 0 | 1 | 0 | 838.3062 | 838.3065 | 19.67 | 1.6303 | 0.0145 | 0.0046 | 0.0079 | 0.0207 | 0.0047 | 0.0050 |
| 4 | 1 | 0 | 0 | 0 | 871.3170 | 871.317 | 24.87 | 1.6261 | 0.8018 | 0.6293 | 0.5162 | 0.5523 | 0.4032 | 0.3598 |
| 4 | 1 | 0 | 0 | 0 | 871.3170 | 871.3184 | 14.96 | 16.5201 | 22.4439 | 203566 | 21.1238 | 19.5844 | 19.7912 | 19.6040 |
| 3 | 2 | 0 | 0 | 0 | 912.3436 | 912.3628 | 16.48 | 0.3713 | 0.1556 | 0.3408 | 0.4798 | 0.3457 | 0.3267 | 0.3186 |
| 3 | 2 | 0 | 0 | 0 | 912.3436 | 912.3439 | 13.67 | 1.6481 | 3.1469 | 3.0602 | 3.2325 | 2.5625 | 2.6408 | 2.8845 |
| 5 | 0 | 1 | 0 | 0 | 976.3479 | 976.3497 | 11.13 | 1.4788 | 1.9633 | 2.4280 | 2.1045 | 2.1414 | 2.0217 | 2.0111 |
| 3 | 1 | 0 | 1 | 0 | 1000.3591 | 1000.357 | 24.03 | 0.2053 | 0.0800 | 0.0414 | 0.0408 | 0.0504 | 0.0666 | 0.0621 |
| 3 | 1 | 0 | 1 | 0 | 1000.3591 | 1000.359 | 21.34 | 0.1908 | 0.1631 | 0.3667 | 0.5430 | 0.5062 | 0.7766 | 0.8085 |
| 4 | 1 | 1 | 0 | 0 | 1017.3749 | 1017.374 | 12.9 | 0.1418 | 0.3402 | 0.4111 | 0.7203 | 0.3650 | 0.7169 | 0.5219 |
| 2 | 2 | 0 | 1 | 0 | 1041.3859 | 1041.381 | 23.25 | 0.0744 | 0.0057 | 0.0126 | 0.0017 | 0.0062 | 0.0029 | 0.0045 |
| 2 | 2 | 0 | 1 | 0 | 1041.3859 | 1041.384 | 24.36 | 0.0631 | 0.0010 | 0.0020 | 0.0000 | 0.0034 | 0.0000 | 0.0000 |
| 2 | 2 | 0 | 1 | 0 | 1041.3859 | 1041.385 | 19.5 | 0.6799 | 0.7978 | 0.7485 | 0.8113 | 0.5925 | 0.7501 | 0.7035 |
| 4 | 2 | 0 | 0 | 0 | 1074.3959 | 1074.394 | 19.96 | 0.0482 | 0.0300 | 0.0314 | 0.0422 | 0.0460 | 0.0303 | 0.0300 |
| 4 | 2 | 0 | 0 | 0 | 1074.3959 | 1074.397 | 17.92 | 3.5791 | 4.4022 | 4.5699 | 4.5321 | 4.6143 | 4.4800 | 4.6152 |
| 4 | 1 | 0 | 1 | 0 | 1162.4119 | 1162.41 | 24.24 | 0.2295 | 0.0931 | 0.0385 | 0.0457 | 0.0896 | 0.1241 | 0.0530 |
| 4 | 1 | 0 | 1 | 0 | 1162.4119 | 1162.412 | 24.87 | 0.8838 | 0.5358 | 0.4637 | 0.4846 | 0.4865 | 0.4325 | 0.3767 |
| 4 | 2 | 0 | 1 | 0 | 1365.4909 | 1365.491 | 24.12 | 0.0849 | 0.0517 | 0.1255 | 0.1172 | 0.1605 | 0.1618 | 0.1441 |
| 1 | 1 | 1 | 0 | 0 | 531.2159 | 531.2146 | 26.05 | 0.1097 | 0.1284 | 0.2180 | 0.1777 | 0.1561 | 0.1939 | 0.1872 |

[symbol]

indicates data missing or illegible when filed

Among the OS structures identified, some OS are described for the first time in Thoroughbred mare's milk. Previously reported neutral OS from horse colostrum includeGal(β1-3)Gal(β1-4)Glc, Gal(β1-6)Gal(β1-4)Glc and Gal(β1-3)[Gal(β1-4)GlcNAc(β1-6)]Gal(β1-4)Glc and unusual phosphorylated N-acetlylactosamine. The number of OS identified is higher than reported in the literature so far. Albrecht et al. ] reported in a recent review 37 OS in mare's colostrum [Albrecht, S et al. British Journal of Nutrition 2014, 111, 1313-1328.], and Difilippo et al. reported 16 OS in the mature milk of four breeds, being specially important the difference in presence/absence of OS type 1 [Difilippo, et al. Journal of agricultural and food chemistry 2015, 63, 4805-4814]. Differences in the findings of these studies can be explained by the diverse methodologies used, mare breeds studied and lower abundance of some OS (especially OS type 1).

Neutral OS were the most abundant (58.3%), followed by acidic OS containing Neu5Ac (33.3%), with a minor presence of fucosylated OS structures (6.25%) and only one structure containing NeuGc (2.1%). In comparison, Albrecht et al. reported that acidic OS containing NeuAc were the most abundant (54.5%) followed by neutral (43.2%) and fucosylated (2.7%) [Albrecht, S et al. British Journal of Nutrition 2014, 111, 1313-1328] Difilippo et al.. reported the same ratio of neutral and acidic structures but did not detect any fucosylated OS in mare's milk [Difilippo, et al. Journal of agricultural and food chemistry 2015, 63, 4805-4814].

Thoroughbred mare's milk OS have less structural variety compared with human milk's nearly 200 characterized structures; yet, when comparing the overall OS structural typology and diversity, mare milk contains a higher number of OS with structural features that are uniquely found in human milk and are only found at the trace level in bovine milk. Additionally, OS in Thoroughbred mare's milk are found in greater array of oligosaccharides (48 structures here identified) compared to what described for porcine milk (39 structures) or goat milk (38 structures) Similarly to human and bovine milk, a few OS structures comprised more than 60% of the total OS, in this case, 3_1_0_0_0, 4_1_0_0_1 LNnP-I and 3′-SL. Despite the number of fucosylated structures characterized in mare's milk and in porcine milk being similar, the contribution of these OS to the total was slightly lower (6.25% in mare vs. 9.1% in porcine milk) yet, still higher than that found in bovine milk (1%). Human milk contains both type I (LNT, LNH, LNFP-II) and type II (LNnT, LNnH, LNFP-III) OS, whereas bovine milk has been shown to contain predominantly type II core OS with lower amounts of OS type I. In contrast, mare's milk contained both type I (LNT, LNH) and type II (LNnT, LNnH) OS in considerable amounts.

### Mare's Milk Oligosaccharide Variation During Early Lactation

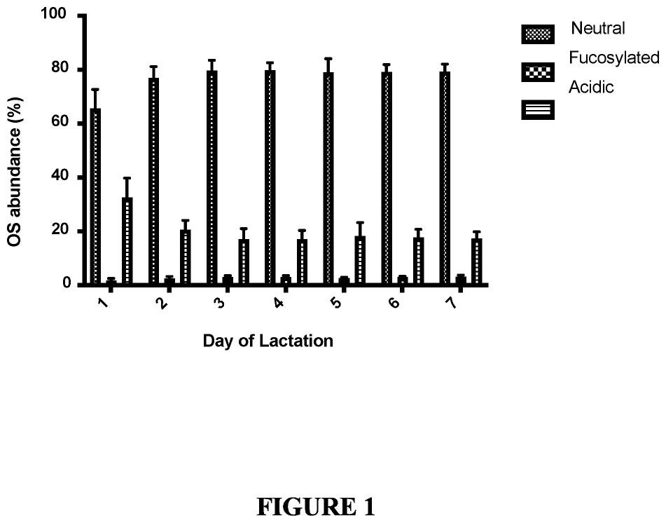

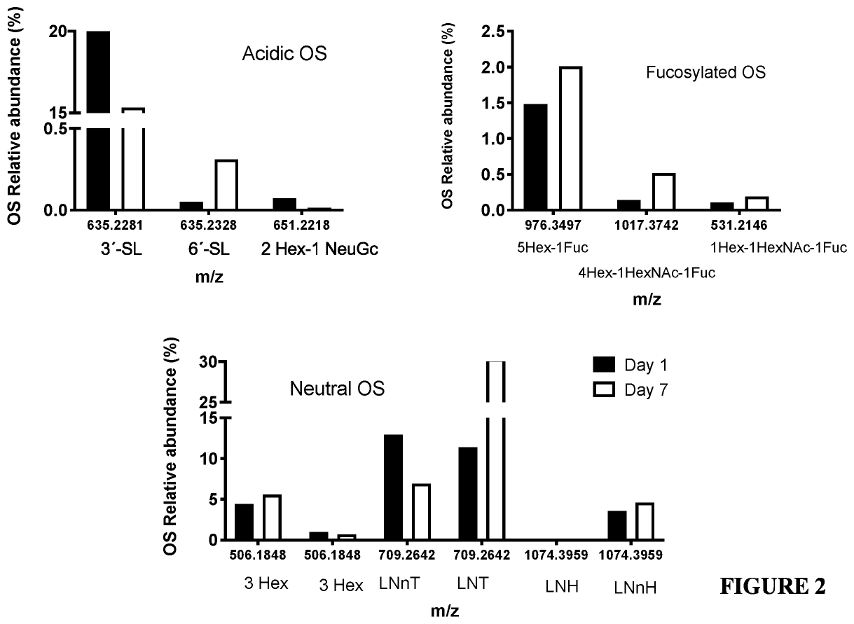

OS class variation was evaluated during the first week of lactation (FIG. 1). Whereas neutral OS increased from day 1 to 2 and remained stable during the rest of period evaluated, acidic OS decreased in the first 2 days and remained stable afterwards. Interestingly, there was a modest but noticeable increase in fucosylated OS at Day 3 (from 1.7% to 3.05%), and their abundance remained stable up to Day 7. These OS variations are further explored in FIG. 2, where the most abundant OS of each class are plotted—3′-SL, the most abundant at Day 1, decreased from 21.6% at Day 1 to 15.3% at Day 7; LNT increased from 11.4% to 33.0%; and the OS with composition 5_0_1_0_0 varied from 1.5% to 2.0%. The single OS structure containing NeuGc decreased with time, with abundance from 0.073% to trace amount (0.014%).

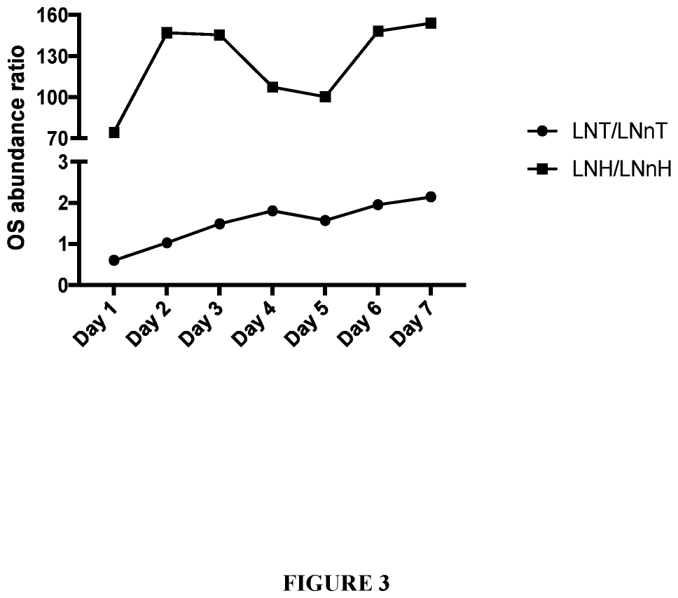

To evaluate the predominant OS core type, the ratio LNT/LNnT and LNH/LNnH was calculated along lactation (FIG. 3). At Day 1, LNnT was predominant over LNT (ratio <1), which was opposite to the ratio of LNH and LNnH (ratio >1); however, the ratio of type I/type II OS increased with lactation in both comparisons, suggesting mare's milk composition becomes relatively closer to that of human milk as lactation advances as indicated by its higher content of type I OS and the marginal increase in fucosylated OS.

To fully characterize the OS in mare's milk, this dataset was further analyzed by High-Performance Anion-Exchange Chromatography coupled with Pulsed Electrochemical Detection (HPAEC-PAD) to measure the concentration of lactose and OS during the first week of lactation. To date, the commercially available OS standards at the necessary purity are scarce, limiting the number of structures quantifiable compared with the OS identified by nano LC-Chip QToF MS/MS. Ten OS, as well as lactose, were monitored for concentration over the first week of lactation (FIG. 3). The most abundant OS were 3′-SL, 6′-SLN and 6′-SL, whereas for neutral OS, LNT was predominant, followed by LNnT and 3 Hexose and 2 Hex-1HexNAc. The lactose content was in the range 18-24 g/L, a value lower than that described for human milk (60-65 g/L) and bovine milk (44-52 g/L).

The concentration of total OS at lactation Day 1 was 217 mg/L, whereas it decreased throughout lactation; 117 mg/L at Day4 and 79 mg/L at Day 7 (Table 2).

When the individual concentrations of specific OS were evaluated, different trends were observed depending the OS considered. The apparent high standard deviation did not derive from measurement issues, but rather was the result of the natural basal level of OS in the 4 animals considered. Regardless of the variation in concentration, some trends were observed in all the four animals. For example, while 3′-SL, LNT and LNnT continuously decreased with time, 6′-SL and 6′-SLN increased, 2 Hex-1HexNAc remained stable until Day 6, and 3 Hex which was low abundant became undetectable at Day 7 (Table 2). Independently of the individual OS variation, there was a net decrease in the total neutral and acidic OS with time—total acidic OS sharply decreased during the first days while neutral OS remained stable for 6 days. Lactose content increased during the first week of lactation from 18.2 g/L at Day 1 to 24.0 g/L at Day 7 of lactation. This trend is also observed in human and bovine milk, showing a well-known increase during the first weeks of lactation]. However, mare's milk had a lower lactose concentration when compared with other mammal milks.

TABLE 2
Quantification of OS in mare's milk (mg/L) during the first
seven days of lactation. Results expressed as average ± standard deviation (n = 3) for each one of the 4 animals.

| | [Oligosaccharides] (mg/L) | | | | | | | |
|---|---|---|---|---|---|---|---|---|
| RT | 2 Hex-1HexNAc | LNnT | 3 Hex | LNT | 6′-SLN | 6′-SL | 3′-SL | |
| (min) | 4.33 | 5.87 | 7.79 | 8.7 | 26.64 | 29.13 | 31.08 | Total OS |
| Day 1 | 0.504 ± 0.401 | 1.165 ± 1.185 | 0.971 ± 0.471 | 8.928 ± 8.728 | 11.719 ± 4.5 | 7.126 ± 3.922 | 187.531 ± 126.1 | 217.815 ± 131.302 |
| Day 2 | 0.745 ± 0.48 | 1.026 ± 0.44 | 0.871 ± 0.515 | 6.892 ± 3.982 | 19.638 ± 7.5 | 16.848 ± 1.241 | 105.327 ± 33.333 | 151.108 ± 35.307 |
| Day 3 | 0.703 ± 0.406 | 0.695 ± 0.413 | 0.751 ± 0.424 | 8.86 ± 6.09 | 18.044 ± 5.903 | 18.737 ± 4.408 | 79.381 ± 39.553 | 127.151 ± 43.395 |
| Day 4 | 0.681 ± 0.255 | 0.529 ± 0.2 | 0.975 ± 0.3 | 6.656 ± 4.217 | 18.781 ± 3.877 | 18.189 ± 2.295 | 71.526 ± 25.97 | 117.161 ± 40.115 |
| Day 5 | 0.777 ± 0.594 | 0.403 ± 0.096 | 0.827 ± 0.186 | 8.174 ± 4.6 | 18.789 ± 6.053 | 15.75 ± 2.604 | 50.318 ± 15.632 | 94.625 ± 23.204 |
| Day 6 | 0.715 ± 0.290 | 0.367 ± 0.146 | 0.648 ± 0.293 | 7.194 ± 2.638 | 15.86 ± 5.052 | 15.635 ± 4.482 | 48.983 ± 18.914 | 89.078 ± 26.266 |
| Day 7 | 0.953 ± 0.87 | 0.273 ± 0.09 | 0 ± 0 | 2.367 ± 2.047 | 13.984 ± 3.137 | 13.382 ± 5.789 | 48.793 ± 37.598 | 79.753 ± 40.833 |

Comparison of Mare's Milk OS with other Mammalian Milks

Unique OS are synthesized only by certain species (e.g. human milk contains unique fucosylated OS) due to the different genetic, metabolic and lactation-specific synthetic pathways. This work demonstrated that Thoroughbred mare's milk shares eight OS structures with human, bovine, pig and goat milk (3′sialyllactose, 6′sialyllactose, 3 Hexose, LNnH, LNH, LNT, LNnT and OS with composition 3 Hex-1 Neu5Ac), but it also contains seven specific OS not reported in other mammal milks (Table 3). The highest number of shared OS structures is with porcine milk (29), followed by bovine milk (28) and goat milk (26). However, when compared with human milk OS composition, there is a higher number of OS shared between human and Thoroughbred mare milk (19) than between human and porcine (13) or bovine milks (11).

TABLE 3
Oligosaccharides in mare, human, bovine, goat and porcine
milks. Note: OS m/z 531.2159 is double charged.

| | Exact | | Presence in | | | |
|---|---|---|---|---|---|---|
| OS | mass | RT | Human | Bovine | Goat | Porcine |
| composition | (calc) | (min) | milk | milk | milk | milk |
| 3_0_0_0_0 | 506.1848 | 10.49 | ✓ | ✓ | ✓ | ✓ |
| 3_0_0_0_0 | 506.1848 | 13.94 | ✓ | ✓ | ✓ | ✓ |
| 3_0_0_0_0 | 506.1848 | 25.48 | ✓ | ϰ | ✓ | ϰ |
| 3_0_0_0_0 | 506.1848 | 15.89 | ✓ | ✓ | ✓ | ✓ |
| 2_1_0_0_0 | 547.2113 | 13.69 | ϰ | ✓ | ✓ | ✓ |
| 2_1_0_0_0 | 547.2113 | 14.96 | ϰ | ✓ | ✓ | ✓ |
| 2_1_0_0_0 | 547.2113 | 14.37 | ϰ | ✓ | ϰ | ✓ |
| 2_1_0_0_0 | 547.2113 | 11.14 | ϰ | ✓ | ϰ | ✓ |
| 2_0_0_1_0 | 635.2274 | 18.94 | ✓ | ✓ | ✓ | ✓ |
| 2_0_0_1_0 | 635.2274 | 12.42 | ✓ | ✓ | ✓ | ✓ |
| 2_0_0_0_1 | 651.2218 | 18.3 | ϰ | ✓ | ϰ | ✓ |
| 4_0_0_0_0 | 668.2376 | 12.77 | ϰ | ✓ | ✓ | ✓ |
| 4_0_0_0_0 | 668.2376 | 14.42 | ϰ | ✓ | ✓ | ✓ |
| 4_0_0_0_0 | 668.2376 | 16.07 | ϰ | ✓ | ✓ | ϰ |
| 1_1_0_1_0 | 676.2534 | 19.49 | ✓ | ✓ | ✓ | ✓ |
| 3_1_0_0_0 | 709.2642 | 17.98 | ϰ | ϰ | ✓ | ✓ |
| 3_1_0_0_0 | 709.2642 | 24.87 | ϰ | ϰ | ✓ | ✓ |
| 3_1_0_0_0 | 709.2642 | 14.96 | ✓ | ✓ | ✓ | ✓ |
| 3_1_0_0_0 | 709.2642 | 11.84 | ✓ | ✓ | ✓ | ✓ |
| 3_1_0_0_0 | 709.2642 | 14.2 | ϰ | ✓ | ✓ | ✓ |
| 2_2_0_0_0 | 750.2907 | 16.16 | ϰ | ϰ | ✓ | ϰ |
| 2_2_0_0_0 | 750.2907 | 13.53 | ϰ | ✓ | ✓ | ✓ |
| 2_2_0_0_0 | 750.2907 | 24 | ϰ | ϰ | ϰ | ϰ |
| 2_2_0_0_0 | 750.2907 | 19.55 | ϰ | ϰ | ✓ | ϰ |
| 2_2_0_0_0 | 750.2907 | 12.6 | ϰ | ✓ | ϰ | ϰ |
| 3_2_0_1_0 | 797.2802 | 21.51 | ✓ | ✓ | ✓ | ✓ |
| 3_0_0_1_0 | 797.2802 | 19.44 | ✓ | ϰ | ϰ | ϰ |
| 5_0_0_0_0 | 830.2905 | 13.42 | ✓ | ✓ | ϰ | ϰ |
| 2_1_0_1_0 | 838.3062 | 21.98 | ϰ | ϰ | ✓ | ✓ |
| 2_1_0_1_0 | 838.3062 | 21.5 | ϰ | ϰ | ϰ | ϰ |
| 2_1_0_1_0 | 838.3062 | 19.67 | ϰ | ϰ | ϰ | ϰ |
| 4_1_0_0_0 | 871.3170 | 24.87 | ϰ | ϰ | ϰ | ✓ |
| 4_1_0_0_0 | 871.3170 | 14.96 | ϰ | ✓ | ✓ | ϰ |
| 3_2_0_0_0 | 912.3436 | 16.48 | ϰ | ✓ | ϰ | ✓ |
| 3_2_0_0_0 | 912.3436 | 13.67 | ϰ | ✓ | ✓ | ✓ |
| 5_0_0_1_0 | 976.3479 | 11.13 | ϰ | ϰ | ϰ | ϰ |
| 3_1_0_1_0 | 1000.3591 | 24.03 | ✓ | ϰ | ϰ | ✓ |
| 3_1_0_1_0 | 1000.3591 | 21.34 | ✓ | ✓ | ϰ | ✓ |
| 4_1_1_0_0 | 1017.3749 | 12.9 | ✓ | ✓ | ϰ | ϰ |
| 2_2_0_1_0 | 1041.3859 | 23.25 | ϰ | ϰ | ϰ | ϰ |
| 2_2_0_1_0 | 1041.3859 | 24.36 | ϰ | ϰ | ϰ | ϰ |
| 2_2_0_1_0 | 1041.3859 | 19.5 | ϰ | ✓ | ϰ | ϰ |
| 4_2_0_0_0 | 1074.3959 | 19.96 | ✓ | ✓ | ✓ | ✓ |
| 4_2_0_0_0 | 1074.3959 | 17.92 | ✓ | ϰ | ϰ | ✓ |
| 4_1_0_1_0 | 1162.4119 | 24.14 | ✓ | ϰ | ϰ | ϰ |
| 4_1_0_1_0 | 1162.4119 | 24.87 | ✓ | ϰ | ϰ | ϰ |
| 4_2_0_1_0 | 1365.4909 | 24.12 | ϰ | ϰ | ✓ | ✓ |
| 1_1_1_0_0 | 531.2159 | 26.05 | ϰ | ϰ | ϰ | ϰ |

TABLE 4
Oligosaccharides unique to mare's milk.

| Hex | HexNAc | Fuc | Neu5Ac | Neu5Gc | Mass | RT |
|---|---|---|---|---|---|---|
| 2 | 2 | 0 | 0 | 0 | 750.2907 | 24 |
| 2 | 1 | 0 | 1 | 0 | 838.3062 | 21.5 |
| 2 | 1 | 0 | 1 | 0 | 838.3062 | 19.67 |
| 5 | 0 | 0 | 1 | 0 | 976.3479 | 11.13 |
| 2 | 2 | 0 | 1 | 0 | 1041.386 | 23.25 |
| 2 | 2 | 0 | 1 | 0 | 1041.386 | 24.36 |
| 1 | 1 | 1 | 0 | 0 | 531.2159 | 26.05 |

| Legend | | Examples |
|---|---|---|
| Hex | Hexose | Glucose/galactose |
| HexNAc | N-acetylhexosamine | N-acetyl-glucosamine/ |
| Fuc | Fucose | N-acetyl- |
| Neu5Ac | N-acetylneuraminic acid | galactosamine |
| Neu5Gc | N-acetylglycolylneuraminic | Fucose |
| | acid | Sialic acids |
| | | Sialic acids |

Compositionally, Thoroughbred mare's milk represents a rich source of milk OS for the neonatal foal. A total of 48 OS structures (including isomers and anomers), corresponding to 20 unique compositions have been identified. Among those, 7 OS were unique for mare milk and were not previously found in other milks. Neutral and, to a certain extent, fucosylated OS increased during the lactation period, whereas acidic OS decreased. The total OS concentration ranged from 217.8 mg/L on day 1 to 79.8 mg/L on day 7. Overall, OS in Thoroughbred mare's milk are compositionally distinct from other mammalian milk OS, with a higher number of OS shared with human milk than with other domestic animals. All documents cited herein are incorporated by reference in their entirety as if each were incorporated individually. These documents exemplify the state of the art at the time of this invention.

All documents cited herein are incorporated by reference in their entirety as if each were incorporated individually. These documents exemplify the state of the art at the time of this invention.

1. A composition comprising an oligosaccharide selected from at least one of 2Hex, 2HexNAc; 2Hex, 1HexNAc 1Neu5Ac; 2Hex, 1HexNAc 1Neu5Ac; 5Hex 1Neu5Ac; 2Hex 2HexNAc 1 Neu5Ac; 2Hex 2HexNAc 1 Neu5Ac; 1Hex 1HexNAc 1 Fuc; Gal(β1-3)Gal(β1-4)Glc; Gal(β1-6)Gal(β1-4)Glc; Gal(β1-3)[Gal(β1-4)GlcNAc(β1-6)]Gal(β1-4)Glc.

2. The composition of claim 1 further comprising at least one bacterial species selected from Bifidobacterium, Lactobacillus or Pedicoccus.

3. A composition of claim 2 wherein the Bifidobacterium species may be selected from Bifidobacterium adolescentis, Bifidobacterium animalis, (e.g., Bifidobacterium animalis subsp. animalis, Bifidobacterium animalis subsp. lactis), Bifidobacterium bifidum, Bifidobacterium breve, Bifidobacterium catenulatum, Bifidobacterium longum (e.g., Bifidobacterium longum subsp. suis, Bifidobacterium longum subsp. infantis (B. infantis) or Bifidobacterium longum subsp. longum), Bifidobacterium pseudocatanulatum, Bifidobacterium pseudolongum, or wherein the Lactobacillus species is selected from Lactobacillus acidophilus, Lactobacillus antri, Lactobacillus brevis, Lactobacillus casei, Lactobacillus coleohominis, Lactobacillus crispatus, Lactobacillus curvatus, Lactobacillus fermentum, Lactobacillus gasseri, Lactobacillus johnsonii, Lactobacillus mucosae, Lactobacillus pentosus, Lactobacillus plantarum, Lactobacillus reuteri, Lactobacillus rhamnosus, Lactobacillus sakei, Lactobacillus salivarius, Lactobacillus paracasei, Lactobacillus kisonensis., Lactobacillus paralimentarius, Lactobacillus perolens, Lactobacillus apis, Lactobacillus ghanensis, Lactobacillus dextrinicus, Lactobacillus shenzenensis, Lactobacillus harbinensis, Lactobacillus equi, or wherein the Pediococcus is selected from Pediococcus parvulus, Pediococcus lolii, Pediococcus acidilactici, Pediococcus argentinicus, Pediococcus claussenii, Pediococcus pentosaceus, or Pediococcus stilesii.

4. (canceled)

5. The composition of claim 1, wherein the B. infantis is activated.

6-9. (canceled)

10. The composition of claim 1 further comprising an oligosaccharides selected from lacto-N-biose (LNB), N-acetyl lactosamine, lacto-N-triose, lacto-N-tetraose (LNT), lacto-N-neotetraose (LNnT), fucosyllactose (FL), lacto-N-fucopentaose (LNFP),lactodifucotetraose, (LDFT) sialyllactose (SL), disialyllacto-N-tetraose (DSLNT), 2′-fucosyllactose (2FL), 3′-sialyllactosamine (3SLN), 3′-fucosyllactose (3FL), 3′-sialyl-3-fucosyllactose(3S3FL), 3′-sialyllactose (3SL), 6′-sialyllactosamine (6SLN), 6′-sialyllactose (6SL), difucosyllactose (DFL), lacto-N-fucopentaose I (LNFPI), lacto-N-fucopentaose II (LNFPII), lacto-N-fucopentaose III (LNFPIII), lacto-N-fucopentaose V (LNFPV), sialyllacto-N-tetraose (SLNT), their derivatives, or combinations thereof

11. A composition of claim 10 wherein the oligosaccharides may include: (a) include one or more Type II oligosaccharide core where representative species include LNnT; (b) one or more oligosaccharides containing the Type II core and GOS, FOS or XOS in 1:5 to 5:1 ratio; (c) one or more oligosaccharides containing the Type II core and 2FL in 1:5 to 5:1 ratio; (d) a combination of (a), (b), and/or (c); (e) include one or more Type I oligosaccharide core where representative species include LNT (f) one or more Type I core and GOS in 1:5 to 5:1 ratio; (g) one or more Type I core and 2FL in 1:5 to 5:1 ratio; and/or (h) a combination of any of (a) to (g) that includes both a type I and type II core.

12. The composition of claim 1 wherein the composition is a nutritional composition.

13. A method of promoting mammalian health comprising administering a composition comprising 2Hex, 2HexNAc; 2Hex, 1HexNAc 1Neu5Ac; 2Hex, 1HexNAc 1Neu5Ac; 5Hex 1Neu5Ac; 2Hex 2HexNAc 1 Neu5Ac; 2Hex 2HexNAc 1 Neu5Ac; 1Hex 1HexNAc 1 Fuc Gal(β1-3)Gal(β1-4)Glc, Gal(β1-6)Gal(β1-4)Glc and/or Gal(β1-3)[Gal(β1-4)GlcNAc(β1-6)]Gal(β1-4)Glc to a mammal.

14. A method of claim 13 further comprising administering at least one bacterial species selected from Bifidobacterium, Lactobacillus or Pedicoccus to the mammal.

15. The method of claim 14 wherein the administered Bifidobacterium species may be selected from Bifidobacterium adolescentis, Bifidobacterium animalis, (e.g., Bifidobacterium animalis subsp. animalis, Bifidobacterium animalis subsp. lactis), Bifidobacterium bifidum, Bifidobacterium breve, Bifidobacterium catenulatum, Bifidobacterium longum (e.g., Bifidobacterium longum subsp. suis, Bifidobacterium longum subsp. infantis (B. infantis) or Bifidobacterium longum subsp. longum), Bifidobacterium pseudocatanulatum, Bifidobacterium pseudolongum, or wherein the administered Lactobacillus species is selected from Lactobacillus acidophilus, Lactobacillus antri, Lactobacillus brevis, Lactobacillus casei, Lactobacillus coleohominis, Lactobacillus crispatus, Lactobacillus curvatus, Lactobacillus fermentum, Lactobacillus gasseri, Lactobacillus johnsonii, Lactobacillus mucosae, Lactobacillus pentosus, Lactobacillus plantarum, Lactobacillus reuteri, Lactobacillus rhamnosus, Lactobacillus sakei, Lactobacillus salivarius, Lactobacillus paracasei, Lactobacillus kisonensis ., Lactobacillus paralimentarius, Lactobacillus perolens, Lactobacillus apis, Lactobacillus ghanensis, Lactobacillus dextrinicus, Lactobacillus shenzenensis, Lactobacillus harbinensis, Lactobacillus equi, or wherein the administered Pediococcus is selected from Pediococcus parvulus, Pediococcus lolii, Pediococcus acidilactici, Pediococcus argentinicus, Pediococcus claussenii, Pediococcus pentosaceus, or Pediococcus stilesii.

16. (canceled)

17. A method of claim 14, wherein the B. infantis is activated.

18.-21. (canceled)

22. The method of claim 13, wherein the composition administered further comprises an oligosaccharides selected from lacto-N-biose (LNB), N-acetyl lactosamine, lacto-N-triose, lacto-N-tetraose (LNT), lacto-N-neotetraose (LNnT), fucosyllactose (FL), lacto-N-fucopentaose (LNFP), lactodifucotetraose, (LDFT) sialyllactose (SL), disialyllacto-N-tetraose (DSLNT), 2′-fucosyllactose (2FL), 3′-sialyllactosamine (3SLN), 3′-fucosyllactose (3FL), 3′-sialyl-3-fucosyllactose(3S3FL), 3′-sialyllactose (3SL), 6′-sialyllactosamine (6SLN), 6′-sialyllactose (6SL), difucosyllactose (DFL), lacto-N-fucopentaose I (LNFPI), lacto-N-fucopentaose II (LNFPII), lacto-N-fucopentaose III (LNFPIII), lacto-N-fucopentaose V (LNFPV), sialyllacto-N-tetraose (SLNT), their derivatives, or combinations thereof.

23. The method of claim 13, wherein the oligosaccharides may include: (a) include one or more Type II oligosaccharide core where representative species include LNnT; (b) one or more oligosaccharides containing the Type II core and GOS in 1:5 to 5:1 ratio; (c) one or more oligosaccharides containing the Type II core and 2FL in 1:5 to 5:1 ratio; (d) a combination of (a), (b), and/or (c); (e) include one or more Type I oligosaccharide core where representative species include LNT (f) one or more Type I core and GOS in 1:5 to 5:1 ratio; (g) one or more Type I core and 2FL in 1:5 to 5:1 ratio; and/or (h) a combination of any of (a) to (g) that includes both a type I and type II core.

24. The method of claim 13, further comprising administering to the mammal a nutritive composition for mammalian neonates, comprising milk oligosaccharides found in (a) mammalian milk, preferably in equine, human or bovine milk, and/or (b) equine milk and optionally (c) a bacteria.

25. The method of claim 24, wherein the milk oligosaccharides are provided by inclusion in the nutritive composition one or more of human milk, bovine milk, infant formula, follow on formula, weaning foods, baby foods, meal replacers, feeds and feed supplements, beverages, post-surgery recovery drinks, in a powder form, which may optionally be suspended in oil, to be added to conventional foods or beverages.

26. The method of claim 13, wherein the administered composition is used increase the abundance of Bifidobacterium in the intestine of the mammal.

27. A method of claim 26, wherein the composition further reduces the abundance of pathogens in the intestines of the mammal.

28. A method of claim 26, wherein the composition reduces stool pH.

29. A method of claim 26, wherein the composition reduces inflammation in the mammal.

30. A method of claim 26, wherein the composition is used to reduce symptoms of autoimmune diseases in the mammal.
